(19) United States
(12) Patent Application Publication
(10) Pub. No.: US 2023/0190856 A1
(43) Pub. Date: Jun. 22, 2023

(54) COMPOSITION COMPRISING A COMPLEX COMPRISING A CURCUMINOID COMPOUND, AND STEVIOL GLYCOSIDES OR A LICORICE EXTRACT OR A FRACTION THEREOF, AND USES THEREOF

(71) Applicants: KOREA RESEARCH INSTITUTE OF BIOSCIENCE AND BIOTECHNOLOGY, Daejeon (KR); BIOTEN CO., LTD, Jeongeup-si (KR)

(72) Inventors: Woo Song LEE, Daejeon (KR); Hyung-Jun Kwon, Daejeon (KR); Woo Sik Kim, Daejeon (KR); Young Bae Ryu, Daejeon (KR); In Chul Lee, Daejeon (KR); Hyung Jae Jeong, Daejeon (KR); Ji Young Park, Daejeon (KR)

(73) Assignees: KOREA RESEARCH INSTITUTE OF BIOSCIENCE AND BIOTECHNOLOGY, Daejeon (KR); BIOTEN CO., LTD, Jeongeup-si (KR)

(21) Appl. No.: 18/067,805

(22) Filed: Dec. 19, 2022

(30) Foreign Application Priority Data
Dec. 17, 2021 (KR) 10-2021-0181905
Aug. 17, 2022 (KR) 10-2022-0102795

(51) Int. Cl. A61K 36/9066; A61K 36/28; A61K 36/484; A61P 31/14; A61K 9/00

(52) U.S. Cl. CPC A61K 36/9066; A61K 36/28; A61K 36/484; A61P 31/14; A61K 9/0056

(57) ABSTRACT

The present disclosure relates to a composition comprising a complex that includes a curcuminoid-based compound and a steviol glycoside, or a curcuminoid-based compound and a licorice extract or a fraction thereof; and a use of the composition for enhancement of immunity according to an enhancement of activity or a cell number of immune cells and/or a use of the composition for prevention, improvement, or treatment of COVID-19.

The complex including curcumin and a steviol glycoside, or a licorice extract or a fraction thereof according to the present disclosure, which are food materials that have been registered with the Ministry of Food and Drug Safety and have received the Generally Recognized as Safe (GRAS) rating from the U.S. FDA, and whose safety has already been confirmed, inhibits symptoms of COVID-19 by activating Th1 cells, CD8 T cells, and NK cells. Therefore, the complex can be provided as a food and pharmaceutical composition for preventing, improving, or treating COVID-19.

### TECHNICAL FIELD

The present disclosure relates to a composition comprising a complex that includes a curcuminoid-based compound and a steviol glycoside, or a curcuminoid-based compound and a licorice extract or a fraction thereof; and a use of the composition for enhancement of immunity according to an enhancement of activity or a cell number of immune cells and/or a use of the composition for prevention, improvement, or treatment of COVID-19.

### BACKGROUND ART

Severe acute respiratory syndrome coronavirus-2 (SARS-CoV-2, 2019-nCoV), which is the cause of the COVID-19 epidemic, first occurred in Wuhan, China in December 2019, and has since spread to China and the entire world. In response, the World Health Organization (WHO) declared a Public Health Emergency of International Concern (PHEIC) with regard to COVID-19, and COVID-19 was declared a global pandemic on Mar. 11, 2020, following the Hong Kong flu (1968) and a novel swine flu (2009). COVID-19 is transmitted when droplets (saliva droplets) of an infected person penetrate the respiratory tract or mucous membranes of the eyes, nose, or mouth. Once infected, after an incubation period of about 2 to 14 days, respiratory symptoms such as fever, cough, and dyspnea or pneumonia appear as the main symptoms, and although rare, cases of asymptomatic infection have also been reported.

To date, Korean Patent Application Publication No. 10-2021-01 28829 and U.S. Pat. No. 11,191,827 B1 have been disclosed as technologies for preventing, improving, or treating COVID-19; additionally, relevant results have been continuously released. However, immediate application of these technologies as a therapeutic agent is difficult in most cases due to safety and efficacy issues.

### DISCLOSURE

### Technical Problem

An object of the present disclosure is to provide a method for preventing or treating COVID-19, in which the method includes administering a pharmaceutical composition for preventing or treating COVID-19 comprising, as an active ingredient, a complex including a curcuminoid-based compound or a pharmaceutically acceptable salt thereof; and a steviol glycoside or a pharmaceutically acceptable salt thereof, or a licorice extract or a fraction thereof to a subject.

Another object of the present disclosure is to provide a method for enhancing immunity, in which the method includes administering a composition for enhancing immunity containing, as an active ingredient, a complex including a curcuminoid-based compound or a pharmaceutically acceptable salt thereof; and a steviol glycoside or a pharmaceutically acceptable salt thereof, or a licorice extract or a fraction thereof to a subject.

Still another object of the present disclosure is to provide a pharmaceutical composition for preventing or treating COVID-19 comprising, as an active ingredient, a complex including a curcuminoid-based compound or a pharmaceutically acceptable salt thereof; and a steviol glycoside or a pharmaceutically acceptable salt thereof, or a licorice extract or a fraction thereof.

Still another object of the present disclosure is to provide a composition for enhancing immunity comprising, as an active ingredient, a complex including a curcuminoid-based compound or a pharmaceutically acceptable salt thereof; and a steviol glycoside or a pharmaceutically acceptable salt thereof, or a licorice extract or a fraction thereof.

Still another object of the present disclosure is to provide a food composition for preventing or improving COVID-19 comprising, as an active ingredient, a complex including a curcuminoid-based compound or a sitologically acceptable salt thereof; and a steviol glycoside or a sitologically acceptable salt thereof, or a licorice extract or a fraction thereof.

Still another object of the present disclosure is to provide a food composition for enhancing immunity comprising, as an active ingredient, a complex including a curcuminoid-based compound or a sitologically acceptable salt thereof; and a steviol glycoside or a sitologically acceptable salt thereof, or a licorice extract or a fraction thereof.

### Technical Solution

Respective descriptions and embodiments disclosed in the present disclosure may also be applied to other descriptions and embodiments. Further, the scope of the present disclosure is not limited by the specific description below.

An aspect of the present disclosure provides a method for preventing or treating COVID-19, in which the method includes administering a pharmaceutical composition for preventing or treating COVID-19 comprising, as an active ingredient, a complex including a curcuminoid-based compound represented by the following Formula 1 or a pharmaceutically acceptable salt thereof; and a steviol glycoside or a pharmaceutically acceptable salt thereof, or a licorice extract or a fraction thereof to a subject.

The curcuminoid-based compound of the present disclosure may be represented by the following Formula 1.

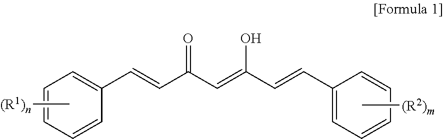

In Formula 1 above, R¹ and R² are each independently hydrogen, a hydroxyl group, or a C₁₋₁₀ alkoxy group, and n and m are 1≤n≤5 and 1≤m≤5.

In an embodiment, in Formula 1 above, R¹ may be hydrogen, a hydroxyl group, or a C₁₋₁₀ alkoxy group independently of R², and R² may be hydrogen, a hydroxyl group, or a C₁₋₁₀ alkoxy group independently of R¹.

Additionally, in R¹ where n≥2, R¹ may be hydrogen, a hydroxyl group, or a C₁₋₁₀ alkoxy group independently, and R² may be hydrogen, a hydroxyl group, or a C₁₋₁₀ alkoxy group independently.

In another embodiment, the curcuminoid-based compound represented by Formula 1 of the present disclosure may be a compound represented by any one of the following Formulas 2 to 4 or a mixture thereof, but is not limited thereto.

The following Formula 2 may be curcumin, the following Formula 3 may be demethoxycurcumin, which is a natural analogue/congener of curcumin, and the following Formula 4 may be bisdemethoxycurcumin, which is a natural analogue/congener of curcumin.

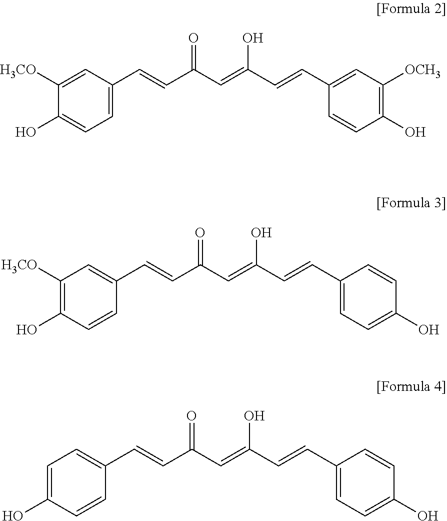 [SMILES: COc1cc(/C=C/C(=O)/C=C(O)/C=C/c2ccc(O)c(OC)c2)ccc1O ; COc1cc(/C=C/C(=O)/C=C(O)/C=C/c2ccc(O)cc2)ccc1O ; O=C(/C=C(O)/C=C/c1ccc(O)cc1)/C=C/c1ccc(O)cc1]

In an embodiment of a mixture of the compounds represented by any one selected from Formulas 2 to 4 above, the weight ratio of curcumin:demethoxycurcumin:bisdemethoxycurcumin included in the mixture may be 50 to 98:1 to 25:1 to 25 [w/w], 60 to 90:5 to 20:5 to 20 [w/w] or 70 to 80:10 to 15:10 to 15 [w/w] based on the total weight of 100, but is not limited thereto.

In the present disclosure, the curcuminoid-based compound may be used interchangeably with a turmeric pigment. In addition, curcumin, demethoxycurcumin, bisdemethoxycurcumin, or a combination thereof may also be used interchangeably with a turmeric pigment.

In another embodiment, the curcuminoid-based compound represented by Formula 1 of the present disclosure may be obtained by extracting dry rhizome of Curcuma longa Linné or turmeric with ethanol, oil, or an organic solvent (Ministry of Food and Drug Safety (Korea), Food and Food Additives Code), but the extraction method is not limited thereto. In particular, the curcuminoid-based compound may be extracted in the form of curcumin, demethoxycurcumin, bisdemethoxycurcumin, or a combination thereof, but is not limited thereto.

In the present disclosure, it is apparent to those skilled in the art that the curcuminoid-based compound of the present disclosure may include all of the compounds belonging to the curcuminoid-based compound instead of merely including curcumin, demethoxycurcumin, and bisdemethoxycurcumin described above, and that the derivatives or isomers of curcumin, demethoxycurcumin, and bisdemethoxycurcumin can also be used.

As used herein, the term “derivative” refers to a compound obtained by substituting a part of the structure of the compound above with another atom or atomic group.

As used herein, the term “isomer” refers to a compound that has the same molecular formula, but does not have the same linkage method or spatial arrangement of constituent atoms in a molecule. The isomer includes, for example, structural isomers and stereoisomers.

As the above-mentioned curcuminoid-based compounds, analogs, derivatives, and isomers thereof, those commercially available may be purchased and used, or those extracted and isolated from the plants such as turmeric and curcuma that have been collected or cultivated in nature may be used, but are not limited thereto.

Additionally, in the present disclosure, a plant extract containing a curcuminoid-based compound represented by Formula 1 above or a pharmaceutically acceptable salt thereof may be used, for example, a turmeric extract, a curcuma extract, etc. may be used, but the plant extract to be used in the present disclosure not limited thereto.

In the present disclosure, as the turmeric extract, curcuma extract, turmeric or curcuma, those commercially available may be purchased and used, or those extracted and isolated from the plants that have been collected or cultivated in nature may be used, but are not limited thereto.

As used herein, the term “steviol glycoside” refers to a natural polysaccharide substance derived from Stevia (Stevia rebaudiana Bertoni), with a 300-fold higher sweetness than sugar. The steviol glycoside consists of various sugars such as steviol glycosides A1 and A2, steviols 1, 2, and 3, dulcoside, and rebaudiosides a, b, c, e, and f.

In the present disclosure, steviol glycoside may preferably be derived from Stevia (Stevia rebaudiana Bertoni).

In an embodiment, the steviol glycoside may be obtained from the leaf part of stevia, but is not limited thereto.

In another embodiment, the steviol glycoside may be obtained by treating a water-soluble extract, which was obtained by hot-water extraction of dried leaves, with adsorption resin followed by concentration, purification (e.g., recrystallization) using methanol or ethanol, and drying (Ministry of Food and Drug Safety (Korea), Food and Food Additives Code), but the extraction method is not limited thereto.

In addition, in the present disclosure, a plant extract containing steviol glycoside may be used, and for example, a stevia extract, etc. may be used, but is not limited thereto.

In the present disclosure, as the steviol glycoside, stevia extract, or stevia, those commercially available may be purchased and used, or those extracted and isolated from the plants that have been collected or cultivated in nature may be used, but are not limited thereto.

As used herein, “licorice” is a perennial herbaceous plant belonging to the family Leguminosae, and is native to or cultivated in northern China, Siberia, southern Italy, Manchuria, Mongolia, and Uzbekistan. The licorice may be, for example, Glycyrrhiza inflata BATALIN, Glycyrrhiza uralensis FISCHER, Glycyrrhiza glabra LINNE, etc., but is not limited thereto.

As used herein, the term “extract” includes a liquid extract itself and an extract of any formulation, which may be formed using the liquid extract, such as a liquid extract obtained by extracting the licorice, a diluted or concentrated solution of the liquid extract, a dry product obtained by drying the liquid extract, a crude purified product or purified product of the liquid extract, or a mixture thereof.

As a method of preparing the licorice extract, a common extraction method in the art, such as ultrasonic extraction, filtration, and reflux extraction may be used. The licorice extract may preferably be an extract obtained by extracting a dry product of licorice obtained by grinding licorice, from which foreign substances are removed by washing and drying, with water, a C₁₋₄ alcohol, or a mixed solvent thereof, more preferably an extract obtained by extracting with a C₁₋₄ alcohol, and most preferably an extract obtained by extracting with methanol or ethanol. In particular, the extraction solvent is preferably used in an amount of 2 to 20 times the dry weight of licorice. For example, the dry product of licorice is minced and placed in an extraction vessel, and a C₁₋₄ lower alcohol or a mixed solvent thereof (preferably methanol or ethanol) is added thereto and left at room temperature for a predetermined period of time, and then the resultant is filtered to obtain an alcohol extract. In particular, it is preferred that the resultant be left at room temperature for one week, and optionally, an additional step such as concentration or freeze-drying may be performed thereafter, but the extraction method is not limited thereto. Alternatively, a commercially available licorice extract may be purchased and used.

As used herein, the term “fraction” refers to a product obtained by a fractionation method of separating a specific component or a specific group from a mixture containing various components. With regard to the licorice fraction, a polar solvent fraction and a non-polar solvent fraction may be obtained respectively, for example, by suspending the licorice extract in water and then performing fractionation using a non-polar solvent such as hexane or ethyl acetate. Specifically, the fraction may be obtained by suspending the crude extract of licorice in distilled water, and then adding a non-polar solvent such as hexane or ethyl acetate with a volume of about 1 to 100 times, preferably about 1 to 5 times the volume of the suspension, and extracting and separating a non-polar solvent soluble layer over 1 to 10 times, preferably 2 to 5 times, but the method to obtain the fraction is not limited thereto. Further, a common fractionation process may be additionally performed (Harborne, J. B. Phytochemical methods: A guide to modern techniques of plant analysis, 3rd Ed. pp. 6-7, 1998).

In an embodiment, the complex of the present disclosure may include i) a curcuminoid-based compound represented by Formula 1 above or a pharmaceutically acceptable salt thereof; and a steviol glycoside or a pharmaceutically acceptable salt thereof; or ii) a curcuminoid-based compound represented by Formula 1 above or a pharmaceutically acceptable salt thereof; and a licorice extract or a fraction thereof. However, the complex of the present disclosure does not exclude a plant extract including the curcuminoid-based compound or a pharmaceutically acceptable salt thereof; a plant extract including a steviol glycoside or a pharmaceutically acceptable salt thereof, or a fraction thereof.

That is, the complex of the present disclosure may include i) a curcuminoid-based compound represented by Formula 1 above or a pharmaceutically acceptable salt thereof, a plant extract including the same, or a fraction thereof; and a steviol glycoside or a pharmaceutically acceptable salt thereof, a plant extract including the same, or a fraction thereof; or ii) a curcuminoid-based compound represented by Formula 1 above or a pharmaceutically acceptable salt thereof, a plant extract including the same, or a fraction thereof; and a licorice extract or a fraction thereof.

In another embodiment, the complex of the present disclosure may arbitrarily select the ratio between the curcuminoid-based compound represented by Formula 1 above and a steviol glycoside or licorice extract. The weight ratio of these components may be arbitrarily selected and the weight ratio of steviol glycoside:a curcuminoid-based compound or a licorice extract:a curcuminoid-based compound included in the complex may be, for example, 90 to 99.9:0.1 to 10 [w/w], 92 to 99.8:0.2 to 8 [w/w], 94 to 99.7:0.3 to 6 [w/w], 96 to 99.6:0.4 to 4 [w/w], 98 to 99.6:0.4 to 2 [w/w], or 98.5 to 99.6:0.4 to 1.5 [w/w] based on the total weight of 100, but is not limited thereto.

Specifically, the complex of the present disclosure may be such that the weight ratio of a steviol glycoside:a curcuminoid-based compound included in the complex is 90 to 99.9:0.1 to 10 [w/w], 92 to 99.7:0.3 to 8 [w/w], 94 to 99.5:0.5 to 6 [w/w], 96 to 99.3:0.7 to 4 [w/w], 98 to 99.1:0.9 to 2 [w/w], or 98.5 to 99:1 to 1.5 [w/w] based on the total weight of 100, but is not limited thereto.

Alternatively, the complex of the present disclosure may be such that the weight ratio of a licorice extract:a curcuminoid-based compound included in the complex is 90 to 99.9:0.1 to 10 [w/w], 92 to 99.8:0.2 to 8 [w/w], 94 to 99.7:0.3 to 6 [w/w], 96 to 99.6:0.4 to 4 [w/w], 98 to 99.6:0.4 to 2 [w/w], or 99 to 99.6:0.4 to 1 [w/w] based on the total weight of 100, but is not limited thereto.

The composition of the present disclosure may have an effect for preventing or treating COVID-19.

As used herein, the term “coronavirus infection (COVID-19)”, which is a disease caused by infection with severe acute respiratory syndrome coronavirus-2 (SARS-CoV-2, 2019-nCoV), is transmitted when droplets (saliva droplets) of an infected person penetrate the respiratory tract or mucous membranes of the eyes, nose, or mouth. Once infected, after an incubation period of about 2 to 14 days, respiratory symptoms such as fever, cough, and dyspnea or pneumonia appear as the main symptoms, and although rare, cases of asymptomatic infection have also been reported.

As used herein, the term “prevention” means all of the actions by which the occurrence of COVID-19 is restrained or delayed by the administration of the composition, and the term “treatment” means all of the actions by which symptoms of PED virus infection have taken a turn for the better or been altered favorably by the administration of the composition.

In an embodiment of the present disclosure, as a result of confirming the improvement rate by a curcumin and steviol glycoside complex (TSP) on lung lesions in a COVID-19 animal model, a TSP group in which TSP containing a curcumin content of 8.9 mg/kg/day (i.e., TSP 78) was administered for four consecutive days and a TSP group in which TSP containing a curcumin content of 25 mg/kg/day (i.e., TSP-218.75) was administered for four consecutive days showed a respective improvement rate of 14.9% and 31.2% on lung lesions without abnormal findings compared to the VC group infected with SARS-CoV-2 (a severe infection model, lung lesions (62%), improvement rate on lung lesions (1%)) (FIG. 13). In contrast, when a complex further containing water-insoluble curcumin (i.e., TGP-C 78, having a curcumin content of 8.9 mg/kg/day) such that the weight ratio of TSP:water-insoluble curcumin included in the complex is 9:1 [w/w] was administered under the same conditions described above, it resulted in a significantly lowered efficacy, thus showing an improvement rate of 6.1% on lung lesions.

Additionally, as a result of confirming the improvement rate by a curcumin and a licorice extract complex (TGP) on lung lesions, a TGP group, in which TGP containing a curcumin content of 3.0 mg mg/kg/day (i.e., TGP 78) was administered for four consecutive days, showed an improvement rate of 20.2% on lung lesions without abnormal findings compared to the VC group infected with SARS-CoV-2, thus confirming that there was a significant improvement on lung lesions (FIG. 14). However, a complex further containing water-insoluble curcumin (i.e., TGP-C 78, having a curcumin content of 3.0 mg/kg/day) such that the weight ratio of TSP : water-insoluble curcumin included in the complex is 9:1 [w/w] was administered under the same conditions described above, it also resulted in a significantly lowered efficacy, thus showing an improvement rate of 15.3% on lung lesions.

That is, the effect of the complex of the present disclosure described above for preventing or treating COVID-19 may be achieved by water-solubilization of curcumin.

In order for a functional substance to exhibit efficacy in the body, it is essential that the substance be absorbed in the blood and maintain its efficacy for a certain period of time. Therefore, for water-solubilization of functional water-insoluble curcumin, in an embodiment of the present disclosure, curcumin was mixed with a steviol glycoside or licorice extract, and thereby a water-solubilized complex of curcumin was prepared using a microwave stirring extractor (Korean Patent No. 10-1436464).

As used herein, the term “water-solubilization” refers to a phenomenon in which the solubility of a substance that is poorly soluble in water increases due to the presence of a substance such as a surfactant. As a method for water-solubilization, a method of solubilizing useful vitamins or hormones to be water-soluble or promoting emulsion polymerization has been widely applied. In the present disclosure, a steviol glycoside or licorice extract was used as a water-soluble agent, and the steviol glycoside or licorice extract from the present disclosure was mixed with curcumin, which is a water-insoluble substance, to thereby allow curcumin to form a water-soluble complex with a structure that is readily soluble in water (or blood).

Particularly, curcumin and a steviol glycoside, or a licorice extract or a fraction thereof constituting the complex of the present disclosure as an active ingredient are food materials that have been registered with the Ministry of Food and Drug Safety (Korea) and have received the Generally Recognized as Safe (GRAS) rating from the U.S. FDA, and their safety has already been confirmed.

In this regard, the composition containing the complex of the present disclosure can be provided as a pharmaceutical composition for preventing or treating COVID-19 without concern for safety.

The pharmaceutical composition of the present invention may further include an appropriate carrier, excipient, or diluent commonly used in the preparation of pharmaceutical compositions. In particular, the amount of the complex included in the pharmaceutical composition as an active ingredient may be in the range of 0.0001 wt % to 10 wt %, and preferably 0.001 wt % to 1 wt %, based on the total weight of the composition, but is not particularly limited to.

The pharmaceutical composition may have any one formulation selected from the group consisting of tablets, pills, powders, granules, capsules, suspensions, solutions for internal use, emulsions, syrups, sterilized aqueous solutions, non-aqueous solvents, suspensions, emulsions, lyophilized preparations, and suppositories, and may have various formulations for oral or parenteral administration. When formulated, the composition of the present invention may be prepared using commonly used diluents or excipients (e.g., fillers, extenders, binders, wetting agents, disintegrants, surfactants, etc.). Solid formulations for oral administration include tablets, pills, powders, granules, capsules, etc., and such solid formulations are prepared by mixing one or more compounds with one or more excipients (e.g., starch, calcium carbonate, sucrose, lactose, gelatin, etc.). In addition to simple excipients, lubricants (e.g., magnesium stearate, talc, etc.) may also be used. Liquid formulations for oral administration include suspensions, solutions for internal use, emulsions, syrups, etc., and may include various excipients (e.g., wetting agents, sweeteners, fragrances, preservatives, etc.), in addition to water and liquid paraffin, which are simple diluents frequently used. Formulations for parenteral administration include sterilized aqueous solutions, non-aqueous solutions, suspensions, emulsions, lyophilized preparations, and suppositories. As non-aqueous solvents or suspending agents, propylene glycol, polyethylene glycol, plant oils (e.g., olive oil), injectable esters (e.g., ethyl oleate, etc.), etc. may be used. As a base of suppositories, witepsol, Macrogol, Tween 61, cacao butter, laurin butter, glycerogelatin, etc. may be used.

The pharmaceutical composition of the present invention may be for oral administration, but is not limited thereto.

The composition of the present invention may be administered in a pharmaceutically effective amount.

As used herein, the term “pharmaceutically effective amount” refers to an amount sufficient to treat diseases at a reasonable benefit/risk ratio applicable to any medical treatment. The effective dose level may be determined depending on factors including a subject's type and severity of illness, age, and sex, type of disease, activity of drugs, sensitivity to drugs, time and route of administration, excretion rate, duration of treatment, drugs used concurrently, and other factors known in the medical field. The composition of the present invention may be administered individually or in combination with other therapeutic agents, and may be administered sequentially or simultaneously with conventional therapeutic agents. Additionally, the composition may be administered in a single dose or multiple doses. It is important to administer the composition in the minimum amount that exhibits the maximum effect without causing side effects, in consideration of all the above-described factors, and this amount may easily be determined by those skilled in the art. A preferred administration dose of the composition of the present invention may vary depending on the patient's condition, body weight, severity of disease, type of drug, and route and period of administration. However, for preferred effects, the composition of the present invention may be administered at a daily dose of 0.0001 mg/kg to 500 mg/kg, preferably 0.001 mg/kg to 250 mg/kg. In an embodiment, the composition may be administered at a daily dose of 25 mg/kg or higher. The composition may be administered once or several times a day. The composition may be administered to various mammals (e.g., rats, livestock, humans, etc.) via various routes. Any administration mode common in the art may be included without limitation (e.g., oral, rectal, intravenous, intramuscular, subcutaneous, intrauterine, and intracerebrovascular injections). Specifically, the composition may be orally administrated, but is not limited thereto.

Additionally, the pharmaceutical composition of the present invention may be used in the form of veterinary pharmaceutical drugs as well as pharmaceutical drugs applied to humans. In particular, the term “animal” used herein has a concept including livestock and pets.

In the present disclosure, the pharmaceutical composition may include a salt, particularly in the form of a pharmaceutically acceptable salt. As the salt, a salt commonly used in the art (e.g., an acid addition salt formed by a pharmaceutically acceptable free acid) may be used without limitation.

As used herein, the term “pharmaceutically acceptable salt” refers to any and all organic or inorganic addition salts of the composition of the present disclosure which have an effective action that is relatively non-toxic and harmless to a subject at such a concentration that the side effects attributable to the salt do not reduce the beneficial effects of the composition of the present disclosure.

Acid addition salts are prepared by conventional methods, for example, by dissolving a compound in an excess amount of an aqueous acid solution and precipitating the salt using a water-miscible organic solvent (e.g., methanol, ethanol, acetone, and acetonitrile). Equimolar amounts of a compound and an acid or alcohol (e.g., glycol monoethyl ether) in water are heated, and then the mixture is evaporated and dried, or the precipitated salt is suction-filtered.

In particular, as the free acid, organic acids and inorganic acids may be used. As the inorganic acids, hydrochloric acid, phosphoric acid, sulfuric acid, nitric acid, etc. may be used; and as the organic acids, methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc. may be used, but the free acid is not limited thereto.

Additionally, the pharmaceutically acceptable metal salt may also be prepared using bases. An alkali metal salt or alkaline earth metal salt may be obtained, for example, by dissolving the compound in an excess amount of an alkali metal hydroxide or alkaline earth metal hydroxide solution, filtering a non-dissolved compound salt, and followed by evaporation and drying of the filtrate. In particular, it is pharmaceutically suitable to prepare sodium, potassium, or calcium salts as a metal salt, but the metal salt is not limited thereto. Further, silver salts corresponding thereto may be obtained by reacting the alkali metal or alkaline earth metal salt with a suitable silver salt (e.g., silver nitrate).

Pharmaceutically acceptable salts of the present disclosure include salts of acidic or basic groups that may be present in the composition of the present disclosure, unless otherwise indicated. For example, pharmaceutically acceptable salts may include sodium, calcium, and potassium salts of hydroxy groups, and other pharmaceutically acceptable salts of amino groups may include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate). These pharmaceutically acceptable salts may be prepared using the preparation methods of salts known in the art.

As used herein, the term “subject” refers to any animal in which COVID-19 has occurred or can occur, and the subject can be effectively treated by administering the pharmaceutical composition of the present disclosure to a subject suspected of having COVID-19.

As used herein, the term “administration” refers to introduction of the pharmaceutical composition of the present disclosure to a subject suspected of having COVID-19 by any suitable method. With regard to the administration route, the pharmaceutical composition of the present disclosure may be administered through various oral or parenteral routes which may reach a target tissue, as described above.

The pharmaceutical composition of the present disclosure may be administered in a pharmaceutically effective amount, as described above.

The pharmaceutical composition of the present disclosure may be applicable to any subject with no particular limitation as long as the purpose is to prevent or treat COVID-19 of the subject. For example, non-human animals (e.g., monkeys, dogs, cats, rabbits, marmots, rats, mice, cows, sheep, pigs, goats, etc.), birds, fish, etc. may be used, and the pharmaceutical composition may be administered parenterally, subcutaneously, intraperitoneally, intrapulmonally, and intranasally, and for local treatment, if necessary, the pharmaceutical composition may be administered by any suitable method including intralesional administration. A preferred dose of the pharmaceutical composition of the present disclosure varies according to the condition and weight of a subject, degree of a disease, type of drug, and route and duration of administration, but may be appropriately selected by those skilled in the art. For example, the pharmaceutical composition may be administered by oral, rectal or intravenous, intramuscular, subcutaneous, intrauterine dural, or intracerebrovascular injection, but the administration route is not limited thereto.

In the present disclosure, the administration may be oral administration, but is not limited thereto.

Another aspect of the present disclosure provides a method for enhancing immunity, in which the method includes administering a composition for enhancing immunity containing, as an active ingredient, a complex including a curcuminoid-based compound represented by Formula 1 above or a pharmaceutically acceptable salt thereof; and a steviol glycoside or a pharmaceutically acceptable salt thereof, or a licorice extract or a fraction thereof to a subject.

The curcuminoid-based compound represented by Formula 1 above, steviol glycoside, pharmaceutically acceptable salt, licorice extract, fraction, subject, and administration are as described above.

In the present disclosure, the curcuminoid-based compound represented by Formula 1 above may be a compound represented by any one selected from Formulas 2 to 4 above or a mixture thereof, and these are as described above.

In the present disclosure, the steviol glycoside may be derived from Stevia rebaudiana Bertoni), but is not limited thereto, and it is as described above.

In the present disclosure, the licorice may be Glycyrrhiza inflata BATALIN, Glycyrrhiza uralensis FISCHER, or Glycyrrhiza glabra LINNE, but is not limited thereto, and it is as described above.

The composition of the present disclosure may have an effect for enhancing immunity.

As used herein, the term “immunity enhancement” means enhancing the activity of a series of in vivo immune responses in order to protect the body from antigens, which are foreign substances such as bacteria, viruses, cancer cells, blood and tissues of other people or animals.

In an embodiment, the immunity enhancement may be an enhancement of the activity of immune cells and/or increase of a cell number, for example, the immunity enhancement may be the enhancement of activity and/or a cell number of Th1 cells, CD8 T cells, and/or NK cells, but is not limited thereto.

In the present disclosure, the Th1 cells may be multifunctional Th1 cells that simultaneously induce IFN-gamma, TNF-alpha, and IL-2, and for example, may be CD3+CD4+ cells that simultaneously induce IFN-gamma, TNF-alpha, and IL-2, but are not limited thereto.

The CD8 T cells may be multifunctional CD8 T cells that simultaneously induce IFN-gamma, TNF-alpha, and IL-2, and for example, may be CD3+CD8+ cells that simultaneously induce IFN-gamma, TNF-alpha, and IL-2, but are not limited thereto.

The NK cells may be activated NK cells, for example, IFN-gamma+CD107a+Granzyme B+ NK cells, but are not limited thereto.

In an embodiment of the present disclosure, as a result of administration of a curcumin and steviol glycoside complex (TSP) or a curcumin and a licorice extract complex (TGP) in an immunocompromised animal model in which an immune decline was induced by the administration of cyclophosphamide (CTX), the following was confirmed: the number of CD4+ and CD8+ T cells reduced by CTX (FIGS. 3 and 4), the percentage of Th1 and activated CD8+ T cells (FIGS. 5 and 6), the percentage of multifunctional Th1 cells or CD8+ T cells (FIGS. 7 and 8), and restoration of the number of activated NK cells (FIGS. 9 and 10).

That is, the effect of the complexes of the present disclosure described above on the prevention or treatment of COVID-19 may be achieved by the effect of immunity enhancement in accordance with the activation of Th1 cells, CD8 T cells, and NK cells by way of water-solubilization of curcumin.

In the present disclosure, the composition of the present disclosure may be for oral administration, but is not limited thereto.

Still another aspect of the present disclosure provides a pharmaceutical composition for preventing or treating COVID-19, which contains, as an active ingredient, a complex including a curcuminoid-based compound or a pharmaceutically acceptable salt thereof; and a steviol glycoside or a pharmaceutically acceptable salt thereof, or a licorice extract or a fraction thereof.

Still another aspect of the present disclosure provides a composition for enhancing immunity, which contains, as an active ingredient, a complex including a curcuminoid-based compound or a pharmaceutically acceptable salt thereof; and a steviol glycoside or a pharmaceutically acceptable salt thereof, or a licorice extract or a fraction thereof.

Still another aspect of the present disclosure provides a method for preparing a pharmaceutical composition for preventing or treating COVID-19, which includes:

a) mixing a first composition containing a curcuminoid-based compound represented by Formula 1 above or a pharmaceutically acceptable salt thereof as an active ingredient; and a second composition containing a steviol glycoside or a pharmaceutically acceptable salt thereof; or a licorice extract or a fraction thereof as an active ingredient; and

b) treating the mixture with microwaves.

The curcuminoid-based compound represented by Formula 1 above, steviol glycoside, pharmaceutically acceptable salt, licorice extract, fraction, and COVID-19 are as described above.

As used herein, the term “microwave” refers to an AC signal with a frequency band between 300 MHz and 300 GHz or a wavelength between 1 m and 1 mm.

The method of treating a microwave may be to perform stirring extraction at 1,000 W to 24,000 W for 10 to 200 minutes, at 3,000 W to 22,000 W for 20 to 160 minutes, at 5,000 W to 20,000 W for 30 to 120 minutes, 7,000 W to 18,000 W for 40 to 100 minutes, or at 10,000 W to 15,000 W for 40 to 80 minutes in a microwave stirring extractor (Korean Patent No. 10-1436464), but the method is not limited thereto.

In addition, after the extraction is completed, the method may further include a step of purifying the extract with a filter paper, but the method is not limited thereto.

Still another aspect of the present disclosure provides a food composition for preventing or improving COVID-19, which contains, as an active ingredient, a complex including a curcuminoid-based compound represented by Formula 1 above or a sitologically acceptable salt thereof; and a steviol glycoside or a sitologically acceptable salt thereof, or a licorice extract or a fraction thereof.

Still another aspect of the present disclosure provides a food composition for enhancing immunity, which contains, as an active ingredient, a complex including a curcuminoid-based compound represented by Formula 1 above or a sitologically acceptable salt thereof; and a steviol glycoside or a sitologically acceptable salt thereof, or a licorice extract or a fraction thereof.

The curcuminoid-based compound represented by Formula 1 above, steviol glycoside, licorice extract and a fraction thereof, and COVID-19 are as described above.

In the present disclosure, the curcuminoid-based compound represented by Formula 1 above may be a compound represented by any one selected from Formulas 2 to 4 above or a mixture thereof, but the curcuminoid-based compound is as described above.

In the present disclosure, the steviol glycoside may be derived from Stevia rebaudiana Bertoni, but the steviol glycoside is not limited thereto and is as described above.

In the present disclosure, the licorice may be Glycyrrhiza inflata BATALIN, Glycyrrhiza uralensis FISCHER, or Glycyrrhiza glabra LINNE, but the licorice is not limited thereto and is as described above.

The food composition of the present disclosure may have an effect of prevention or improvement of COVID-19 and/or enhancement of immunity, and these are as described above.

As used herein, the term “improvement” means all of the actions by which the symptoms of a subject who is suspected of having COVID-19 and has developed COVID-19 been improved or advantageously altered using the composition.

The food composition of the present disclosure may be prepared in the forms of pills, powders, granules, infusions, tablets, capsules or liquids, and the food to be added with the composition of the present disclosure includes various foods, for example, beverages, gums, teas, vitamin complexes, health supplements, etc.

As ingredients that may be included in the food composition of the present disclosure, there is no particular limitation to other ingredients that can be included in the food composition except that active ingredients are included as essential components, and various herbal extracts, food supplements, natural carbohydrates, etc. may be included as additional ingredients like general foods. In the food composition, the amount of the essential ingredient may be appropriately determined in accordance with the purpose of use (prevention, improvement, or therapeutic treatment). In particular, the amounts of the active ingredients included in the composition may be 0.0001 wt % to 10 wt %, specifically 0.001 wt % to 1 wt % based on the total weight of the composition, but are not particularly limited thereto.

In addition, the food supplements may include food additives commonly used in the art, for example, flavoring agents, coloring agents, fillers, stabilizers, etc.

Examples of the natural carbohydrates may include general sugars, such as monosaccharides (e.g., glucose, fructose, etc.); disaccharides (e.g., maltose, sucrose, etc.); and polysaccharides (e.g., dextrin, cyclodextrin, etc.), and sugar alcohols such as xylitol, sorbitol, erythritol, etc. In addition to those described above, as the flavoring agent, natural flavoring agents (e.g., rebaudioside A, glycyrrhizin, etc.) and synthetic flavoring agents (saccharin, aspartame, etc.) may be advantageously used.

In addition, the food composition of the present disclosure may include various nutrients, vitamins, minerals (electrolytes), flavoring agents (e.g., synthetic and natural flavoring agents), coloring agents, and fillers (e.g., cheese, chocolate, etc.), pectic acid and salts thereof, alginic acid and salts thereof, organic acids, a protective colloidal thickener, a pH adjusting agent, a stabilizer, a preservative, glycerin, alcohol, a carbonic acid agent used in carbonated drinks, etc. In addition, the food composition of the present disclosure may include fruit flesh for preparing natural fruit juice and fruit juice beverages and vegetable beverages. These ingredients may be used independently or in combination.

In the present disclosure, the health supplement food may include health functional foods, health foods, etc.

The health functional food is the same term as food for special health use (FoSHU) and refers to food with a high medical/care effect, which is processed such that a bioregulatory function contained therein is effectively exhibited, in addition to nutrition supply. In particular, the term “function (functionality)” means regulation of nutrients or obtaining an effect useful for health uses (e.g., physiological actions) in relation to the structures and functions of the human body.

The food including the food composition of the present disclosure may be prepared by methods which are commonly used in the art, and in the food preparation, the food may be prepared by adding raw materials and ingredients which are commonly added in the art. In addition, the food may be prepared in any formulation without limitation as long as it is recognized as food. The food composition of the present disclosure may be prepared in various forms of formulations, and unlike general drugs, the food composition has an advantage in that it has no side effects that may occur during a long-term administration of drugs because food is used as a raw material, and may have excellent portability.

The food composition of the present disclosure may include a salt, particularly a salt in the form of a sitologically acceptable salt, and the definition and details thereof are the same as those described for the pharmaceutically acceptable salt.

In particular, as described above, curcumin and a steviol glycoside, or a licorice extract or a fraction thereof constituting the complex of the present disclosure as an active ingredient are food materials that have been registered with the Ministry of Food and Drug Safety (Korea) and have received the Generally Recognized as Safe (GRAS) rating from the U.S. FDA, and their safety has already been confirmed. Therefore, these ingredients can be provided as a food composition having a use of preventing or improving COVID-19 without concern for safety.

### Advantageous Effects

The complex of the present disclosure including curcumin and a steviol glycoside, or a licorice extract or a fraction thereof, which have been registered with the Ministry of Food and Drug Safety (Korea) and have received the Generally Recognized as Safe (GRAS) rating as food materials by the U.S. FDA, and whose stability has already been confirmed, inhibits symptoms of COVID-19 by activating Th1 cells, CD8 T cells, and NK cells. Therefore, the complex can be provided as a food and pharmaceutical composition for preventing, improving, or treating COVID-19.

### BRIEF DESCRIPTION OF DRAWINGS

FIG. 1 shows an image illustrating a Tencumin S plus (TSP) complex containing water-solubilized curcumin and a steviol glycoside complex; water-solubilized curcumin; and a Tencumin G plus (TGP) complex containing water-solubilized curcumin and a licorice extract complex in this order, all before vacuum freeze-drying.

FIG. 2 shows a schematic diagram illustrating an experiment performed using an immunocompromised animal model.

FIG. 3 shows results confirming the ability of recovering CD4+ and CD8+ T cells by TSP administration in an immunocompromised animal model.

FIG. 4 shows results confirming the ability of recovering CD4+ and CD8+ T cells by TGP administration in an immunocompromised animal model.

FIG. 5 shows results confirming the ability of recovering Th1 and activated CD8+ T cells by TSP administration in an immunocompromised animal model.

FIG. 6 shows results confirming the ability of recovering Th1 and activated CD8+ T cells by TGP administration in an immunocompromised animal model.

FIG. 7 shows results confirming the ability of recovering multifunctional Th1 or multifunctional CD8+ T cells by TSP administration in an immunocompromised animal model.

FIG. 8 shows results confirming the ability of recovering multifunctional Th1 or multifunctional CD8+ T cells by TGP administration in an immunocompromised animal model.

FIG. 9 shows results confirming the ability of recovering activated NK cells by TSP administration in an immunocompromised animal model.

FIG. 10 shows results confirming the ability of recovering activated NK cells by TGP administration in an immunocompromised animal model.

FIG. 11 shows images illustrating lung tissue collected by autopsy for each test group after TSP administration.

FIG. 12 shows images illustrating lung tissue collected by autopsy for each test group after TGP administration.

FIG. 13 shows a graph illustrating the macroscopic cure rate of lung lesions according to TSP administration. Abbreviations: negative control (NC), virus control (VC), and positive control (PC).

FIG. 14 shows a graph illustrating the macroscopic cure rate of lung lesions according to TGP administration. Abbreviations: negative control (NC), virus control (VC), and positive control (PC).

### DETAILED DESCRIPTION OF THE INVENTION

Hereinafter, the present disclosure will be described in detail through exemplary embodiments. However, these exemplary embodiments are for illustrative purposes of the present disclosure and are not intended to limit the scope of the present disclosure.

### EXAMPLE 1: PREPARATION OF COMPLEX

In order to prepare a Tencumin S plus (hereinafter, “TSP”) complex containing water-solubilized curcumin and a steviol glycoside, 200 g of a steviol glycoside was dissolved in 1 L of water (20% concentration; [v/v]) and then 9 g of a turmeric pigment with a purity of about 95% or more produced in India (curcumin:demethoxycurcumin:bisdemethoxycurcumin=75:15:10 [w/w]) was added thereto. When the turmeric pigment was mixed with an aqueous solution of the steviol glycoside, about 30% to 35% of the turmeric pigment was dissolved, and the dissolved mixture was added into a microwave stirring extractor (Korean Patent No. 10-1436464), extracted with stirring at 12,000 W for 60 minutes, purified with a filter paper, and then vacuum freeze-dried to obtain TSP powder.

The TSP contained less than 200 g of the steviol glycoside and 3.0 g or more of the turmeric pigment per 203 g of the total weight of the powder.

In order to prepare a Tencumin G plus (hereinafter, “TGP”) complex containing water-solubilized curcumin and a licorice extract (Dodam Herb Chinese Medicien Products Co., Ltd., Korea; produced in Uzbekistan), 200 g of a steviol glycoside was dissolved in 1 L of water (20% concentration; [v/v]), and then 3 g of a turmeric pigment with a purity of about 95% or more produced in India (curcumin:demethoxycurcumin:bisdemethoxycurcumin=75:15:10 [w/w]) was added thereto. When the turmeric pigment was mixed with an aqueous solution of the licorice extract, about 30% to 35% of the turmeric pigment was dissolved, and the dissolved mixture was added into a microwave stirring extractor (Korean Patent No. 10-1436464), extracted with stirring at 12,000 W for 60 minutes, purified with a filter paper, and then vacuum freeze-dried to obtain TGP powder.

The TSP contained less than 200 g or less of the licorice extract and more than 1.0 g or more of the turmeric pigment per 201 g of the total weight of the powder.

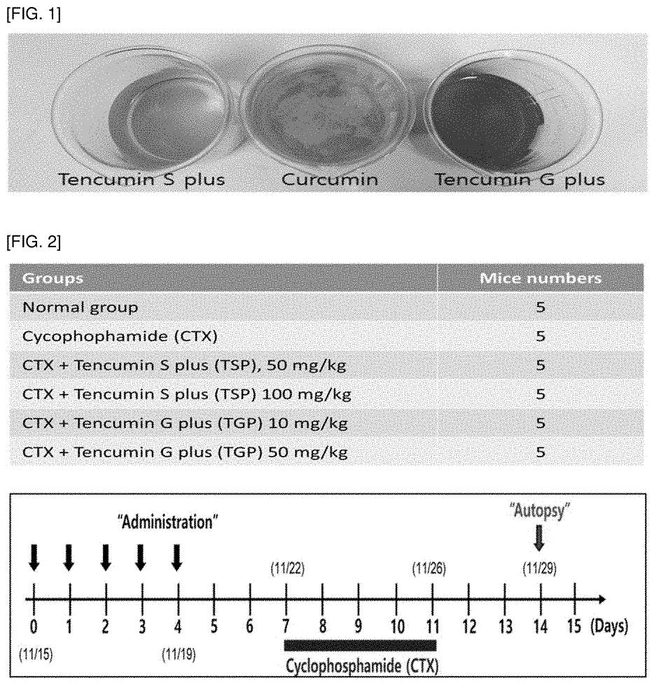

The TSP, curcumin, and TGP before vacuum freeze-drying are as shown in FIG. 1.

### EXAMPLE 2: EVALUATION OF ABILITY OF RECOVERING IMMUNE CELLS BY COMPLEXES IN IMMUNOCOMPROMISED ANIMAL MODEL

### 2-1. Confirmation of Ability of Recovering CD4+ and CD8+ T Cells

TSP (50 mg/kg and 100 mg/kg) or TGP (10 mg/kg and 50 mg/kg) prepared in Example 1 was orally administered to mice once a day for a total of 5 times. Three days thereafter, cyclophosphamide (CTX), which is an immunosuppressant that rapidly reduces the number of T cells in the spleen, was intraperitoneally injected once a day for a total of 5 times (FIG. 2). Three days after the CTX injection, the splenocytes of the mice were separated and treated with a T cell activator (including PMA/Ino; phorbol 12-myristate 13-acetate (PMA), ionomycin, brefeldin A, monensin, and a protein transport inhibitor) for 4 hours.

In order to measure the number of T cells, the splenocytes were treated with live/dead cell staining reagent, anti-CD3, anti-CD4, and anti-CD8 antibodies, left at room temperature for 20 minutes to be stained, washed several times with phosphate-buffered saline (PBS), and then analyzed by flow cytometry.

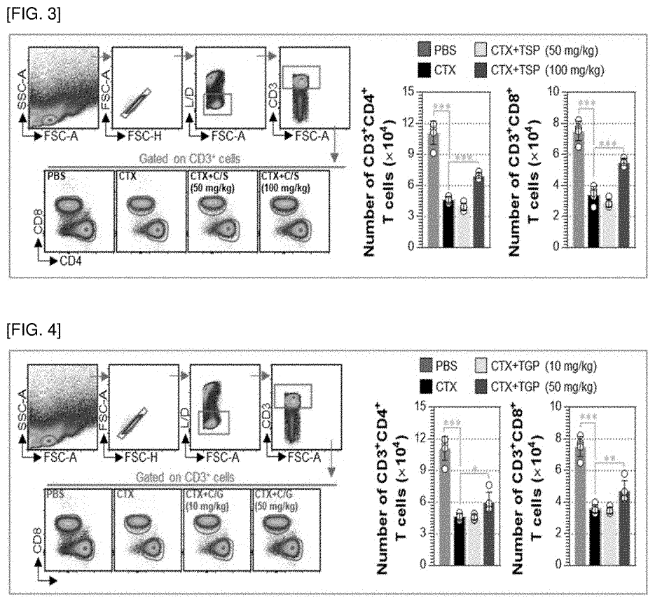

As a result, it was confirmed that when TSP (100 mg/kg) was administered 5 times before the CTX treatment, the number of CD4+ and CD8+ T cells decreased by CTX was recovered (FIG. 3).

Additionally, it was confirmed that when TGP (10 mg/kg or 50 mg/kg) was administered 5 times before the CTX treatment, the number of CD4+ and CD8+ T cells decreased by CTX was recovered (FIG. 4).

### 2-2. Confirmation of Ability of Recovering Th1 and Activated CD8+ T Cells

In order to measure the number of Th1 or activated CD8+ T cells in the same animal model as in Example 2-1 according to the administration of the complex (TSP or TGP) of Example 1, the splenocytes were treated with PMA/Ino for 4 hours, treated with live/dead cell staining reagent, anti-CD3, anti-CD4, anti-CD8, and anti-CD25 antibodies, left at room temperature for 20 minutes to be stained, and washed several times with PBS. While fixing the cell surfaces using the Fix & Perm Cell permeabilization kit, holes were made on the cell surfaces simultaneously, and the cells were left at room temperature for 20 minutes to be stained using anti-IFN-gamma, anti-IL-5, anti-IL-17A, and anti-Foxp3 antibodies, and then analyzed by flow cytometry.

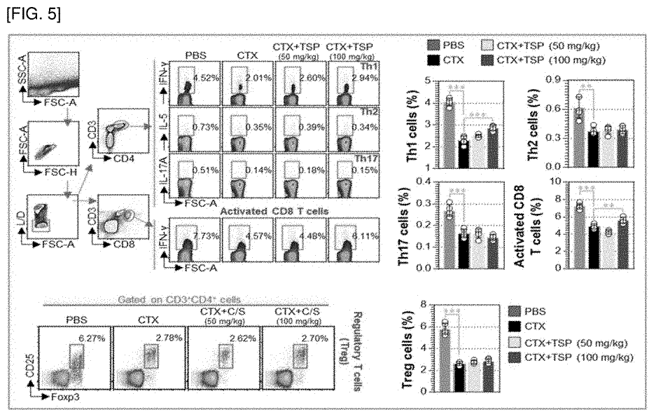

As a result, it was confirmed that when CTX was intraperitoneally injected, all of the number of Th1 cells (IFN-gamma+CD3+CD4+ cells), Th2 cells (IL-5+CD3+CD4+ cells), Th17 cells (IL-17A+CD3+CD4+ cells), regulatory T cells (Foxp3+CD25+CD3+CD4+ cells), and activated CD8+ T cells (IFN-gamma+CD3+CD8+ cells) was reduced, whereas when TSP (100 mg/kg) was administered 5 times before the CTX treatment, the ratio of Th1 cells and activated CD8+ T cells decreased by CTX was recovered (FIG. 5).

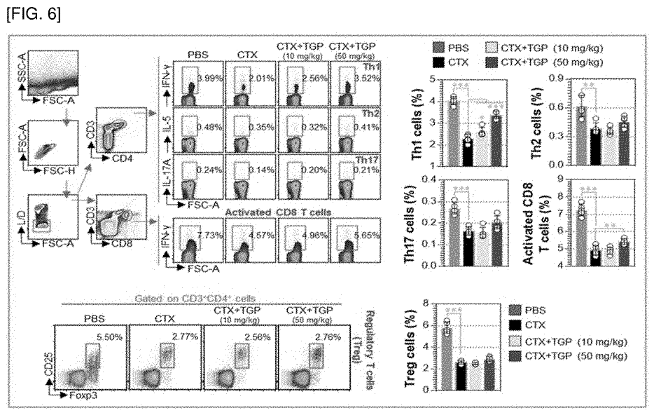

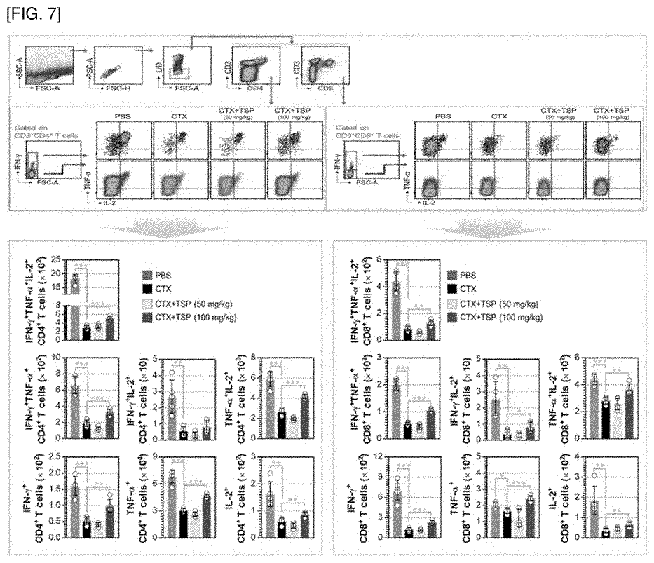

Additionally, it was confirmed that when TGP (10 mg/kg or 50 mg/kg) was administered 5 times before the CTX treatment, the number of Th1 cells and activated CD8+ T cells decreased by CTX was recovered (FIG. 6).

### 2-3. Confirmation of Ability of Recovering Multifunctional Th1 and Multifunctional CD8+ T Cells

CD4+ or CD8+ T cells simultaneously induce IFN-gamma, TNF-alpha, and IL-2, which are Th1 cytokines, and are thus also referred to as multifunctional T cells (multifunctional Th1 or multifunctional CD8+ T cells, respectively).

In order to measure the number of multifunctional T cells according to the administration of the complex (TSP or TGP) of Example 1 in the same animal model as in Example 2-1, the splenocytes were treated with PMA/Ino for 4 hours, treated with live/dead cell staining reagent, anti-CD3, anti-CD4, and anti-CD8 antibodies, left at room temperature for 20 minutes to be stained, and washed several times with PBS. While fixing the cell surfaces using the Fix & Perm Cell permeabilization kit, holes were made on the cell surfaces simultaneously, and the cells were left at room temperature for 20 minutes to be stained using anti-IFN-gamma, anti-TN F-alpha, and anti-IL-2 antibodies, and then analyzed by flow cytometry.

As a result, it was confirmed that when CTX was intraperitoneally injected, the numbers of both multifunctional Th1 cells (CD3+CD4+ cells which simultaneously induce IFN-gamma, TN F-alpha, and IL-2) and multifunctional CD8+ T cells (CD3+CD8+ cells which simultaneously induce IFN-gamma, TN F-alpha, and IL-2) were reduced, whereas when TSP (100 mg/kg) was administered 5 times before the CTX treatment, the ratio of multifunctional Th1 or CD8+ T cells decreased by CTX was recovered (FIG. 7).

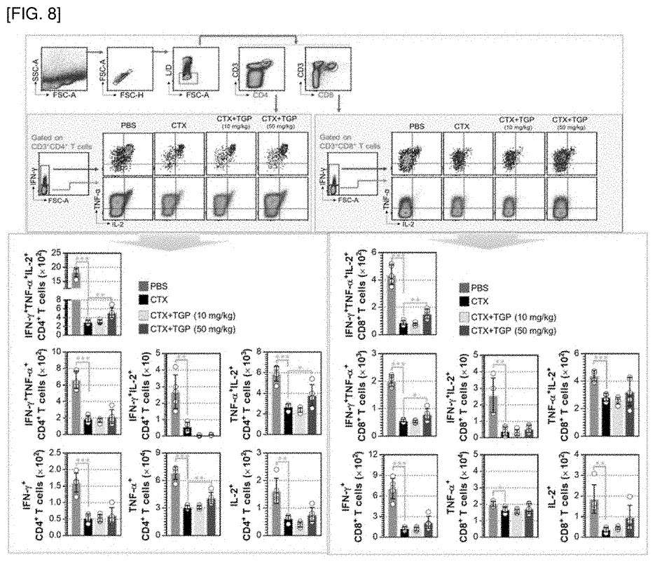

Additionally, it was confirmed that when TGP (50 mg/kg) was administered 5 times before the CTX treatment, the number of multifunctional Th1 cells or CD8+ T cells decreased by CTX was recovered (FIG. 8).

### 2-4. Confirmation of Ability of Recovering NK Cells

In order to measure the number of activated NK cells according to the administration of the complex (TSP or TGP) of Example 1 in the same animal model as in Example 2-1, the splenocytes were treated with PMA/Ino for 4 hours, treated with live/dead cell staining reagent, lineage antibodies (anti-CD19, anti-CD14, and anti-CD3e), and anti-NK1.1 antibodies, and the cells were left at room temperature for 20 minutes to be stained, and were washed several times with PBS. While fixing the cell surfaces using the Fix & Perm Cell permeabilization kit, holes were made on the cell surfaces simultaneously, and the cells were left at room temperature for 20 minutes to be stained using anti-IFN-gamma, anti-CD107a, and anti-Granzyme B antibodies, and then analyzed by flow cytometry.

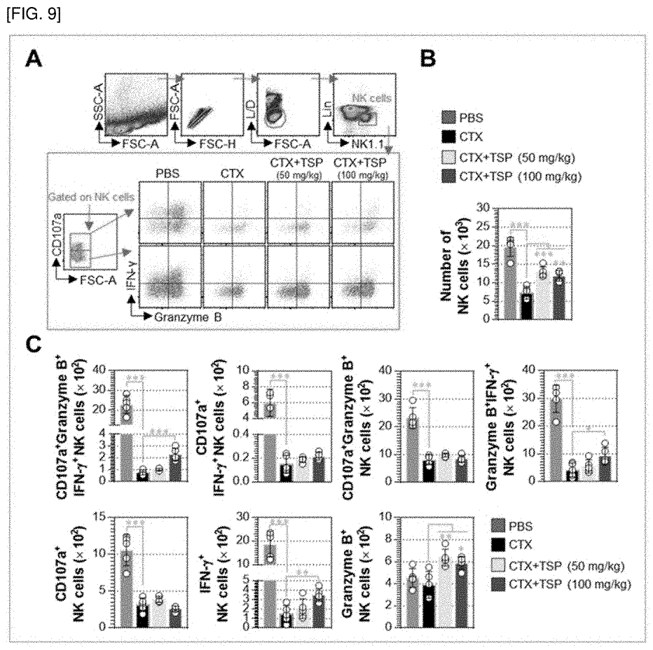

As a result, it was confirmed that when TSP (50 mg/kg or 100 mg/kg) was administered 5 times before the CTX treatment, the number of activated NK cells (IFN-gamma+CD107a+Granzyme B+ NK cells) reduced by CTX was recovered (FIG. 9).

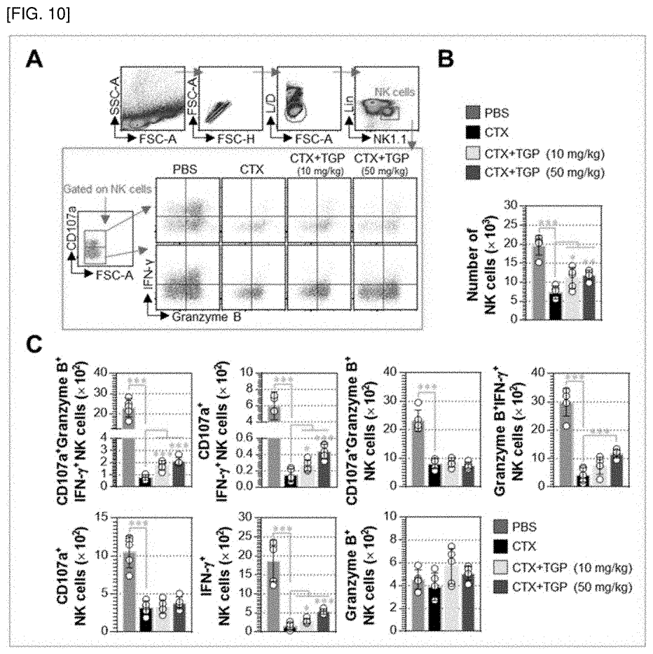

Additionally, it was confirmed that when TGP (10 mg/kg or 50 mg/kg) was administered 5 times before the CTX treatment, the number of NK cells decreased by CTX was recovered (FIG. 10).

According to the results above, it was confirmed that both TSP and TGP have an effect on recovering multifunctional Th1 cells, multifunctional CD8+ T cells, and activated NK cells, all of which are reduced by immunosuppressive agents.

### EXAMPLE 3: EVALUATION OF DRUG EFFICACY IN COVID-19 ANIMAL MODEL

The therapeutic effect of the complex (TSP or TGP) of Example 1 on COVID-19 was confirmed using a Syrian hamster animal model infected with type 2 severe acute respiratory syndrome coronavirus-2 (SARS-CoV-2) and exhibiting COVID-19 symptoms.

The animal model was prepared by administering COVID-19 according to the method shown in Table 1 below, and the negative control group (NC), the positive control virus group (VC), and the test groups administered with TSP were divided as shown in Table 2 below.

Specifically, respiratory anesthesia was performed for 10 minutes using isoflurane. After infecting the hamsters by instilling 100 μL of the prepared SARS-CoV-2 virus into the nasal cavity of the hamsters according to Table 2 below, the drug was orally administered (P.O.) at 10 mL/kg using an oral sonde for each test group.

TABLE 1

| Hamsters | Female, 5 n/Group |
|---|---|
| Age | 11 weeks old |
| Virus Titer | 2.0 × 10⁵ PFU/mL |
| Inoculation Route | intranasal (100 μL) |
| Drug Administration | per oral (P.O., 10 mL/kg) |

TABLE 2

| Test | | | | | |
|---|---|---|---|---|---|
| Group | Group | Dose | Vehicle/Volume | Route/Interval | |
| No. | (n = 5) | (mg/head) | (mL/kg) | (hours) | Remarks |
| 1 | Negative | 0 | water/10 | P.O./(24 | — |
| | Control | | | hours/a total | |
| | (NC) | | | of 4 times) | |
| 2 | Virus | 0 | water/10 | P.O./(24 h/a | — |
| | Control | | | total of 4 | |
| | (VC) | | | times) | |
| 3 | TSP | 218.75 | water/10 | P.O./simultaneous | curcumin |
| | | | (water | administration | content |
| | | | solubilization) | after infection | (25 mg/kg/day) |
| | | | | (24 hours/a | |
| | | | | total of 4 | |
| | | | | times) | |
| 4 | TSP | 39 | water/10 | P.O./simultaneous | curcumin |
| | | | (water | administration | content |
| | | | solubilization) | after infection | (8.9 mg/kg/day) |
| | | | | (24 hours/a | |
| | | | | total of 8 | |
| | | | | times) | |
| 5 | TSP:Curcumin = 9:1 | 39 | water/10 | P.O./simultaneous | curcumin |
| | | | (water | administration | content |
| | | | solubilization) | after infection | (8.9 mg/kg/day) |
| | | | | (24 hours/a | |
| | | | | total of 8 | |
| | | | | times) | |
| 6 | TGP | 39 | water/10 | P.O./simultaneous | curcumin |
| | | | (water | administration | content |
| | | | solubilization) | after infection | (3.0 mg/kg/day) |
| | | | | (24 hours/a | |
| | | | | total of 8 | |
| | | | | times) | |
| 7 | TGP:Curcumin = 9:1 | 39 | water/10 | P.O./simultaneous | curcumin |
| | | | (water | administration | content |
| | | | solubilization) | after infection | (3.0 mg/kg/day) |
| | | | | (24 hours/a | |
| | | | | total of 8 | |
| | | | | times) | |

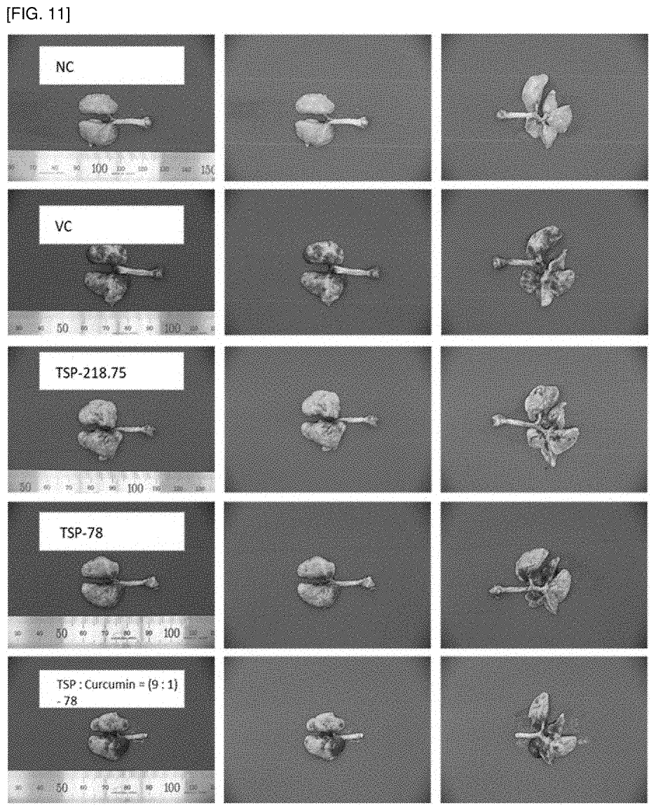

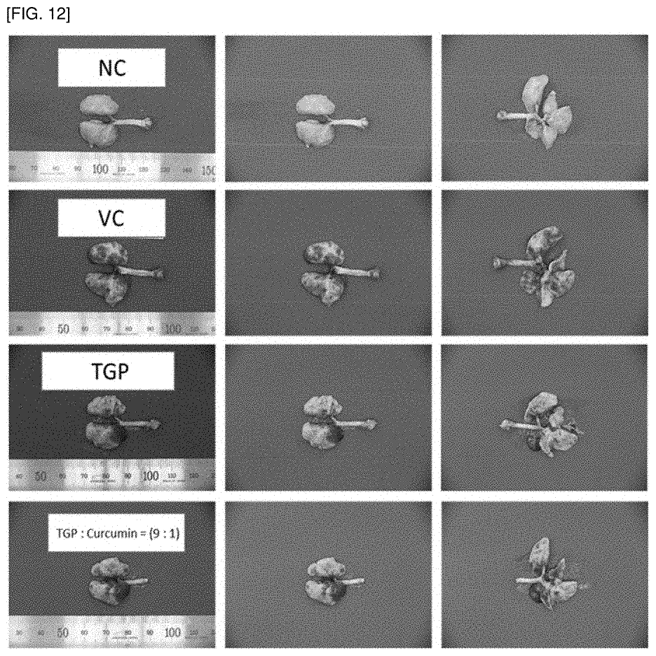

On the 4th day after infection (PID4), macroscopic lung lesions were evaluated for lung tissues collected through autopsy from each test group (FIG. 11 and FIG. 12).

Specifically, each lung tissues were divided into (i) to (v) according to the lung lobe as shown in Table 3 below, and the improvement rate of the lung lesions was visually evaluated according to each weight, and the cure rate of the lung lesions was calculated by substituting the resultant into the following formula.

Macroscopic cure rate of lung lesion=pneumonia incidence rate in TSP group/pneumonia incidence rate in VC group×100

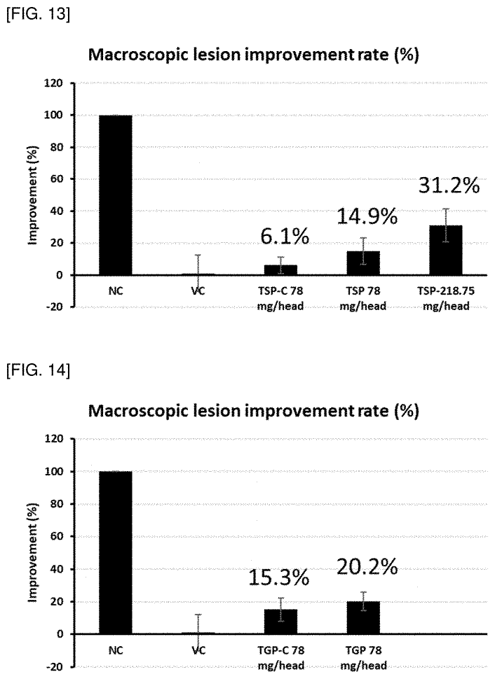

As a result, compared to the VC group infected with SARS-CoV-2 (a severe infection model, lung lesions (62%), an improvement rate of lung lesions (1%)), in TSP groups (TSP 78 and TSP-218.75, respectively), where water-soluble TSP (78 mg/head/day, curcumin content (8.9 mg/kg/day); Test Group 4 in Table 2) or water-soluble TSP (218.75 mg/head/day, curcumin content (25 mg/kg/day); Test Group 3 in Table 2) was administered for 4 consecutive days, the rate of improvement of lung lesions was shown to be 14.9% and 31.2%, respectively, without abnormal findings, and lung lesions were significantly improved. However, when a complex (TSP-C 78; TSP:water-insoluble curcumin=9:1 [w/w]) (78 mg/head/day, curcumin content (8.9 mg/kg/day); Test Group 5 in Table 2) was administered under the same conditions, and an improvement rate of 6.1% of lung lesions was confirmed (FIG. 13).

Additionally, in the TGP group (TGP 78), where water-soluble TSP (78 mg/head/day, curcumin content (3.0 mg/kg/day); Test Group 6 in Table 2) was administered for 4 consecutive days under the same conditions, the rate of improvement in lung lesions was 20.2% without abnormal findings, and thus a significant improvement was confirmed. However, when a complex additionally including water-insoluble curcumin (TGP-C 78; TGP:water-insoluble curcumin=9:1 [w/w]) (78 mg/head/day, curcumin content (3.0 mg/kg/day); Test Group 7 in Table 2) was administered under the same conditions, the efficacy was also significantly lowered, and an improvement rate of 15.3% of lung lesions was confirmed (FIG. 14).

In particular, TSP showed excellent effects of improving bilateral pneumonia, pulmonary edema, and pulmonary hemorrhage caused by SARS-CoV-2 infection.

According to the results above, it was confirmed that when water-insoluble curcumin was additionally added to the complex (TSP or TGP) of Example 1 and orally administered, the efficacy of improving lung lesions was significantly lowered.

From the results of Examples above, it was confirmed that the TSP (which contains water-solubilized curcumin and a steviol glycoside) or the TGP (which contains water-solubilized curcumin and a licorice extract) of the present disclosure enhances the activity and cell number of multifunctional Th1 cells, multifunctional CD8+ T cells, and activated NK cells and significantly improves lung lesions caused by SARS-CoV-2 infection.

In particular, curcumin and a steviol glycoside constituting the TSP, and a licorice extract constituting the TGP of the present disclosure are food materials that have been registered with the Ministry of Food and Drug Safety (Korea) and have received the Generally Recognized as Safe (GRAS) rating from the U.S. FDA, and their safety has already been confirmed. Therefore, these ingredients can be provided as a food and pharmaceutical composition for preventing, improving, or treating COVID-19.

From the foregoing, a skilled person in the art to which the present disclosure pertains will be able to understand that the present disclosure may be embodied in other specific forms without modifying the technical concepts or essential characteristics of the present disclosure. In this regard, the exemplary embodiments disclosed herein are only for illustrative purposes and should not be construed as limiting the scope of the present disclosure. On the contrary, the present disclosure is intended to cover not only the exemplary embodiments but also various alternatives, modifications, equivalents, and other embodiments that may be included within the spirit and scope of the present disclosure as defined by the appended claims.

1. A method for preventing or treating COVID-19, the method comprising administering a pharmaceutical composition for preventing or treating COVID-19 comprising, as an active ingredient, a complex including a curcuminoid-based compound represented by the following Formula 1 or a pharmaceutically acceptable salt thereof; and a steviol glycoside or a pharmaceutically acceptable salt thereof, or a licorice extract or a fraction thereof to a subject: 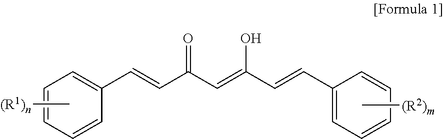
wherein R¹ and R² are each independently hydrogen, a hydroxyl group, or a C₁₋₁₀ alkoxy group, and
n and m are 1≤n≤5 and 1≤m≤5.

2. The method of claim 1, wherein the curcuminoid-based compound represented by the following Formula 1 is a compound represented by any one of the following Formulas 2 to 4 or a mixture thereof:

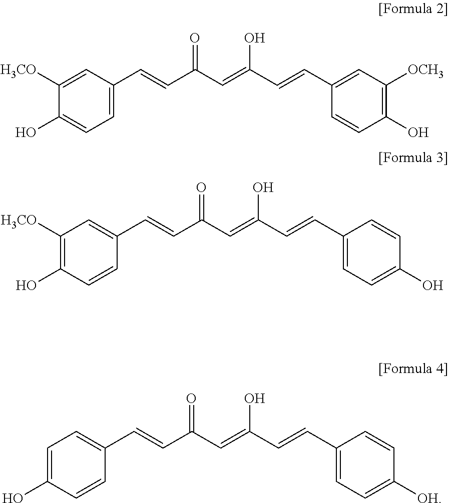 [SMILES: COc1cc(/C=C/C(=O)/C=C(O)/C=C/c2ccc(O)c(OC)c2)ccc1O ; COc1cc(/C=C/C(=O)/C=C(O)/C=C/c2ccc(O)cc2)ccc1O ; O=C(/C=C(O)/C=C/c1ccc(O)cc1)/C=C/c1ccc(O)cc1]

3. The method of claim 1, wherein the steviol glycoside is derived from Stevia rebaudiana Bertoni.

4. The method of claim 1, wherein the licorice is any one or more selected from the group consisting of Glycyrrhiza inflata BATALIN, Glycyrrhiza uralensis FISCHER, and Glycyrrhiza glabra LINNE.

5. The method of claim 1, wherein the composition is for oral administration.

6. A method for enhancing immunity, the method comprising administering a composition for enhancing immunity comprising, as an active ingredient, a complex including a curcuminoid-based compound represented by the following Formula 1 or a pharmaceutically acceptable salt thereof; and a steviol glycoside or a pharmaceutically acceptable salt thereof, or a licorice extract or a fraction thereof to a subject: 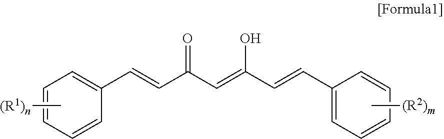
wherein R¹ and R² are each independently hydrogen, a hydroxyl group, or a C₁₋₁₀ alkoxy group, and
n and m are 1≤n≤5 and 1≤m≤5.

7. The method of claim 6, wherein the immunity enhancement is an enhancement of activity or a cell number of any one or more selected from the group consisting of Th1 cells, CD8+ T cells, and NK cells.

8. The method of claim 6, wherein the curcuminoid-based compound represented by Formula 1 above is a compound represented by any one of the following Formulas 2 to 4 or a mixture thereof:

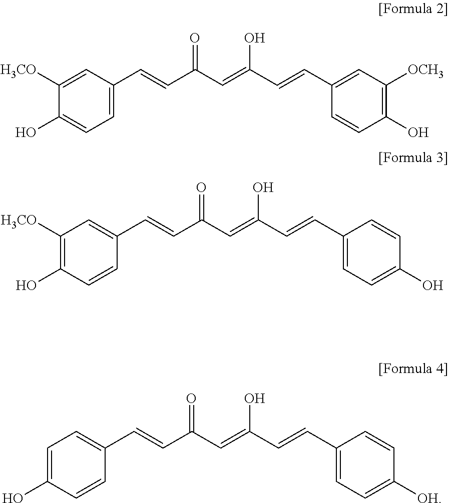 [SMILES: COc1cc(/C=C/C(=O)/C=C(O)/C=C/c2ccc(O)c(OC)c2)ccc1O ; COc1cc(/C=C/C(=O)/C=C(O)/C=C/c2ccc(O)cc2)ccc1O ; O=C(/C=C(O)/C=C/c1ccc(O)cc1)/C=C/c1ccc(O)cc1]

9. The method of claim 6, wherein the steviol glycoside is derived from Stevia rebaudiana Bertoni.

10. The method of claim 6, wherein the licorice is any one or more selected from the group consisting of Glycyrrhiza inflata BATALIN, Glycyrrhiza uralensis FISCHER, and Glycyrrhiza glabra LINNE.

11. The method of claim 6, wherein the composition is for oral administration.
